(19) United States
(12) Patent Application Publication
(10) Pub. No.: US 2008/0028625 A1
(43) Pub. Date: Feb. 7, 2008

(54) Apparatus and Method for Replicating a Plantar Surface of a Foot

(76) Inventors: Willyn Nudelman, Holon (IL); Marina Nudelman-Klepfish, London (GB)

Correspondence Address: NATH & ASSOCIATES, 112 South West Street, Alexandria, VA, 22314, US

(21) Appl. No.: 11/793,222

(22) PCT Filed: Dec. 19, 2005
(86) PCT No.: PCT/IL05/01363

§ 371 Date: Jun. 18, 2007

(30) Foreign Application Priority Data
Dec. 19, 2004 (IL) 165850

(51) Int. Cl. A43D 1/00; A61B 5/00; A61B 5/103

(52) U.S. Cl. 33/515; 33/6; 33/3 A; 33/512; 33/514.2

(57) ABSTRACT

An apparatus and method for replicating the profile of a plantar surface of a foot are provided. It has particular application in the production of footwear insoles, the apparatus comprises a pressure resistant container (2); an elastic membrane (3) sealed to an open face of the container, thereby defining a variable volume chamber within the container; means for reversibly introducing fluid (15) into the chamber; a bed of elongate rods (13) of substantially equal length, in which, in one configuration, the rods can slide freely, one relative to another, along their length; and clamping means (10) for reversibly clamping the bed of rods so that the relative sliding movement of the rods is resisted; the bed of rods being arranged, in use, outside the container so that ends of the rods abut the face of the membrane. In this way, a positive and negative mould of the surface can be formed.

### FIELD OF THE INVENTION

The present invention relates to the field of devices and methods for replicating uneven surfaces, and more particularly, to an apparatus and method for production of personally fitted biomechanical orthopedic insoles.

### BACKGROUND OF THE INVENTION

It is recognized that due to structural deficiencies or deformities of the feet many individuals may suffer from various states of foot pain, and/or physical discomfort ranging from minor back pains to severe disability. Orthopedic insoles can compensate for the structural deficiencies of an individual's foot or position. For individuals suffering from pain in these areas, orthopedic insoles can provide a measure of relief. For others, such as professional athletes and soldiers orthopedic insoles can reduce the chance of injury while improving freedom of motion.

Conventional methods and devices for making orthopedic insoles are based on production of a gypsum foot imprint on which the orthopedic insole is molded. The foot imprint is produced as a rule either (i) for a sole free of load, or (ii) under uncontrolled load conditions—i.e. with an intended user putting weight on the relevant foot.

In the first case, the shape of the foot imprint does not represent its contour under load conditions, and therefore, the distribution of the load on the sole's load-bearing surface appears uneven, because of the differences of the elastic characteristics of the soft tissue and the differences of mobility of the bone elements. Such unevenness creates large difference of specific load for various areas of the sole.

In the second case, the sole is under load either on a hard surface or on an elastic material (see, for example, U.S. Pat. Nos. 4,522,777 to Peterson; 5,387,384 to Irving; and 6,160,264 to Rebiere). The hard surface loading generates large difference of specific load for different areas of the sole, due to the unequal mobility of the bone elements. The consequences of such uneven distribution of pressure in different foot areas are callosities, pain and considerable strains that could result in fracture. In the case of the elastic surface, the distribution of the load depends on the elastic characteristics of the material, the patient's weight and specific pressure on the load-bearing surface of the sole. Selecting elastic materials with fine adjustment of parameters to suit every individual case is impractical, which limits the usage of this method.

### SUMMARY OF THE INVENTION

Despite the known techniques for replicating uneven surfaces, there is a need for a novel apparatus and method for production of insoles based on a loaded foot imprint, taking into consideration the patient's weight and the area of the load-bearing plantar surface of the foot. It would be advantageous to ensure equal distribution of the specific load across the plantar surface of the foot during walking and standing.

In essence, the present invention provides an apparatus for replicating the profile of a plantar surface of a foot that includes the following main components: a device for creating an equally distributed load on a load-bearing part of the surface of the sole under full load with distribution of the load uniformly across the whole surface of the sole; and a device for obtaining an imprint of the load-bearing surface of the sole without need for any intermediate operations.

It should be noted that the terms “plantar surface of a foot” and “sole surface” are synonymous within the description of the present invention, and therefore will be used interchangeably.

Specifically, the apparatus for replicating the profile of the sole surface comprises at least one inflation device configured for creating an equally distributed load on the plantar surface of the foot under full load with distribution of the load uniformly across the whole sole surface, and an imprint-forming device mounted on the top of the inflation device and configured for obtaining an imprint of the plantar surface of the foot under the load.

According to an embodiment of the invention, the inflation device includes: a pressure resistant container; an elastic membrane sealed to an open face of said pressure resistant container, thereby defining a variable volume chamber within the container; a manifold coupled to the pressure resistant container; and a compressor pump configured to provide fluid into the chamber via the manifold, thereby to provide a predetermined pressure within said variable volume chamber, said predetermined pressure required for creating an equally distributed load on said plantar surface of the foot under full load with distribution of the load uniformly across the whole plantar surface.

The imprint-forming device includes a bed of elongate rods having substantially equal length, a clamping unit configured for reversibly clamping the bed of rods so that the relative sliding movement of the rods is resisted, the bed of rods being arranged so that ends of the rods abut the face of said elastic membrane.

The rods may be circular in cross-section, or may have cross-section that more closely tessellates, such as square or hexagonal. The use of a tessellating cross-section minimizes the gaps between the rods, with the beneficial effect that any molding compound used to form a replicate is less likely to seep into the bed of rods.

According to an embodiment of the invention, the apparatus further comprises sensing elements, to sense contact between the elastic membrane and an opposite face of the container resulting from deformation of the membrane, in use.

The apparatus operates by introducing fluid into the chamber until the object (with the soft, uneven surface), such as a foot, is fully supported by the fluid-backed membrane, in this way, an even pressure across the face of the object is ensured. The sensing means acts to provide an indication of when this condition is achieved.

According to an embodiment of the invention, the apparatus further comprises a moveably mounted platform within the cavity, substantially parallel to, and biased towards the membrane. This platform serves, initially to support the object (e.g. a foot), prior to introduction of the fluid. As fluid is introduced into the chamber, the biasing of the platform (e.g. with one or more springs) causes the platform to move with the foot, until the foot is completely supported by the fluid-backed membrane. Preferably, in this embodiment, the device further comprises sensing elements, to sense contact between the elastic membrane and the platform resulting from deformation of the membrane, in use. The sensor element, in this case, may be conveniently located between the platform and the opposite face of the container, and could take the form of a microswitch. Several such sensor elements may be employed—for example, on each corner of the platform—to improve the reliability of the sensing. This serves a similar function to the sensing elements described above.

According to one embodiment of the apparatus, the clamping unit comprises a reversibly deformable tube encircling at least part of the bed of rods, the reversible clamping being achieved by manipulation of the pressure of fluid within the tube, the resultant deformation of which tube squeezes the rods together. Use of an apparatus such as this is particularly advantageous, as the same fluid introduction means as used in the chamber can also be used to drive the clamping unit. In this way, production costs can be minimized.

Alternatively, the clamping unit can comprise an electromagnet acting upon a bed of magnetically susceptible rods. In this way, clamping and unclamping can be carried out rapidly, and without the use of fluid systems, which can leak.

In a particularly advantageous embodiment, the device further comprises a deformable airtight enclosure surrounding the bed of rods, and means to reversibly remove air from within the enclosure. This enclosure—such as a bag—serves two functions: firstly, when it is “vacuum sealed” around the bed of rods, is serves as an additional or alternative clamping mechanism, enabling the clamped bed of rods to be removed from the apparatus for use as a mould. In this way, a number of rod beds may be used with the same apparatus, enabling the apparatus to be used whilst molding is taking place, without needing to replicate the clamping mechanism. Secondly, the enclosure also acts to seal any gaps between the rods down which molding compound might migrate, sticking the rods together and resulting in an uneven surface on the molded item.

For automated and most effective use, the apparatus preferably also comprises control unit to effect the steps of: unclamping the bed of rods (if clamped); then receiving a signal from the sensing means indicating contact between the membrane or with the platform (if present); then introducing fluid into the chamber until receiving a signal from the sensing means indicating loss of contact between the membrane or with the platform (if present); then clamping the bed of rods; then releasing fluid from the chamber.

In this way, when a user (in the case of making shaped insoles) steps onto the device, this is sensed by the sensing means. The control means then starts introducing fluid (e.g. air, from a compressor) into the cavity. When the membrane has “inflated” sufficiently so that the foot is supported completely by the membrane, the sensing elements sense the loss of contact. Thus, the control unit sends commands to stop the fluid introduction, and clamp the rods together. Fluid may now be removed from the chamber to reset the device ready for use.

According to another embodiment of the invention, the apparatus comprises three inflation devices pivotably connected in series to one another and assigned to correct distribution of the different load created by forefoot, midfoot and hindfoot, correspondingly. This embodiment can be used for replicating the profile of a plantar surface of a foot placed in a condition similar to the condition when a foot is in high heel shoes. According to this embodiment, the imprint-forming device of the apparatus includes an upper frame and a lower frame pivotably connected to two lateral walls, thereby allowing tilting of the upper frame and the lower frame with respect to the walls in order to maintain the rods in a vertical position.

Included within the scope of the invention is also a method for replicating the profile of the sole surface using the apparatus of the present invention. The method comprises unclamping the bed of rods, if required, so they slide freely; pressing the plantar surface against the rods, so causing deformation of the elastic membrane; introducing sufficient fluid into the chamber to provide said predetermined pressure within the variable volume chamber; clamping the bed of rods to resist further sliding; using either face of the clamped bed of rods as a mould to form a positive or negative replicate of the surface.

According to one embodiment of the invention, the predetermined value of pressure established in the chamber is such that the membrane deformed owing to the pressure of the foot is not in contact with an opposite face of the chamber.

According to another embodiment of the invention, the predetermined value of pressure established in the chamber is such that the membrane deformed owing to the pressure of the foot is not in contact with the platform.

According to yet another embodiment of the invention, the predetermined value of pressure established in the chamber is evaluated in advance as weight of a user divided by total area of the plantar surface.

A flexible liner may conveniently be laid between the clamped bed of rods and molding compound to prevent ingress of the compound to the bed of rods, and to produce a more smooth surface. Accordingly, when the apparatus also comprises a deformable enclosure, the method also comprises the step of removing air from the airtight enclosure. In this case, the enclosure itself serves the function of the liner, described above.

Also included within the scope of the invention is a replicate of the profile of a soft uneven surface produced according to any of the described methods.

It should be noted that the invention allows production of insoles using a loaded foot imprint, taking into consideration the patient's weight and the area of the load-bearing surface of the foot. This ensures equal distribution of the specific load across the surface of the sole during walking and standing.

There has thus been outlined, rather broadly, the more important features of the invention so that the detailed description thereof that follows hereinafter may be better understood, and the present contribution to the art may be better appreciated. Additional details and advantages of the invention will be set forth in the detailed description.

### BRIEF DESCRIPTION OF THE DRAWINGS

In order to understand the invention and to see how it may be carried out in practice, exemplary embodiments will now be described, by way of non-limiting examples only, with reference to the accompanying drawings, in which:

FIG. 1 illustrates a perspective view showing a partial assembly of the apparatus for replicating the profile of a surface of a sole, according to one embodiment of the invention;

FIG. 2 illustrates a base platform of the apparatus shown in FIG. 1;

FIG. 3 illustrates a device for creating the equally distributed load on the load-bearing surface of the sole under full load with distribution of it uniformly across the whole surface of the sole, according to one embodiment of the invention;

FIG. 4 illustrates a contour-forming device for obtaining positive and/or negative imprint(s) of the load-bearing surface of the sole, according to one embodiment of the invention;

FIG. 5 illustrates schematically a cross-sectional side view of an embodiment of a complete apparatus for replicating the profile of a surface of a sole;

FIG. 6 illustrates an embodiment of the assembly of the apparatus in the course of an initial stage of the insole preparation;

FIG. 7 illustrates the assembly of the apparatus shown in FIG. 6 in the course of a final stage of the insole preparation;

FIG. 8 illustrates a perspective view showing a partial assembly of the apparatus for replicating the profile of a surface of a sole, according to another embodiment of the invention;

FIG. 9 illustrates the assembly of the apparatus shown in FIG. 8 in the course of an initial stage of insole preparation;

FIG. 10 illustrates the assembly of the apparatus shown in FIG. 8 in the course of a final stage of insole preparation;

FIG. 11 illustrates schematically a cross-sectional side view of yet another embodiment of an apparatus for replicating the profile of a surface of a sole;

FIGS. 12A and 12B illustrate schematically a perspective exploded and partially assembled views of an embodiment of an imprint-forming device of the apparatus of the present invention designed for replicating the profile of a plantar surface of a foot placed in a condition similar to the condition when the foot is in high heel shoes;

FIG. 13 illustrates the assembly of the apparatus for replicating the profile of a plantar surface of a foot placed in a condition similar to the condition when the foot is in high heel shoes in the course of a final stage of insole preparation; and

FIG. 14 illustrates a deformable airtight enclosure surrounding the bed of rods, and means to reversibly remove air from within the enclosure.

### DETAILED DESCRIPTION OF EXEMPLARY EMBODIMENTS

The principles and operation of an apparatus for replicating the profile of a surface of a sole according to the present invention may be better understood with reference to the accompanying description and the drawings, in which preferred embodiments of the invention are illustrated. It should be understood that these drawings are given for illustrative purposes only and are not meant to be limiting. The present invention may be embodied in many different forms and should not be construed as limited to the embodiments set forth herein. The same reference numerals and alphabetic characters will be utilized for identifying those components which are common in the surgical fastener and its components shown in the drawings throughout the present description of the invention.

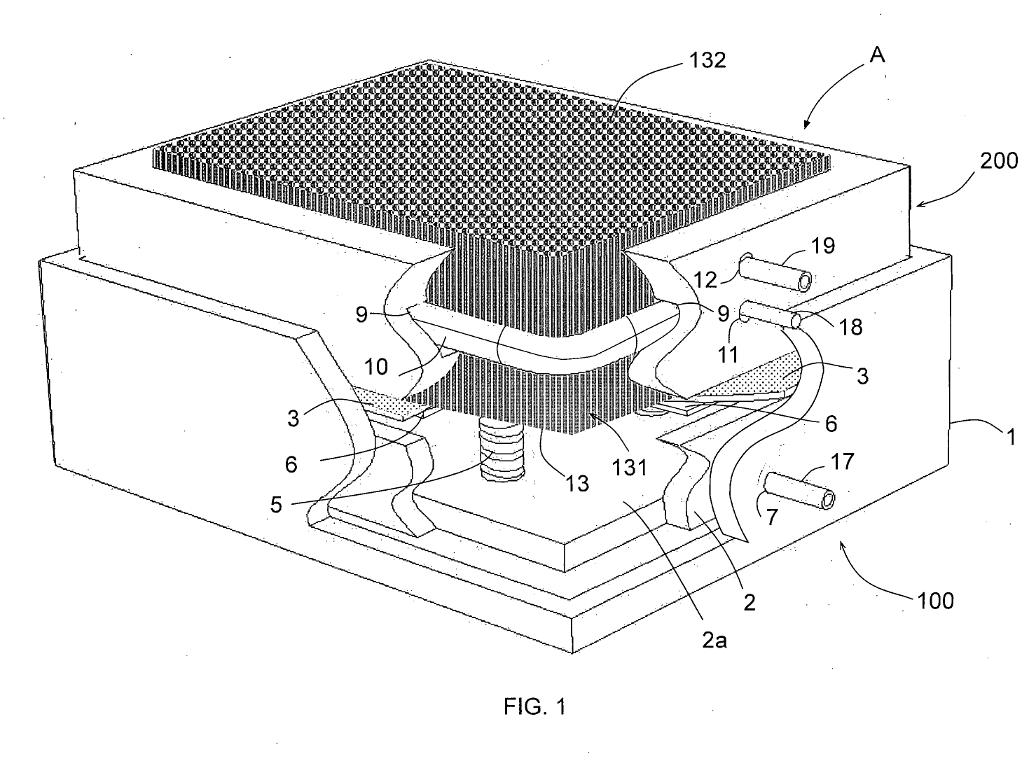

Referring to FIG. 1 through FIG. 5 together, an apparatus A for replicating the profile of a plantar surface of a foot (i.e., surface of the sole) includes an inflation device 100 (shown separately in FIG. 3) for creating an equally distributed load on the plantar surface of the foot under full load with distribution of the load uniformly across the whole sole surface, and an imprint-forming device 200 (shown separately in FIG. 4) for obtaining an imprint of the load-bearing surface of the sole without need for any intermediate operations.

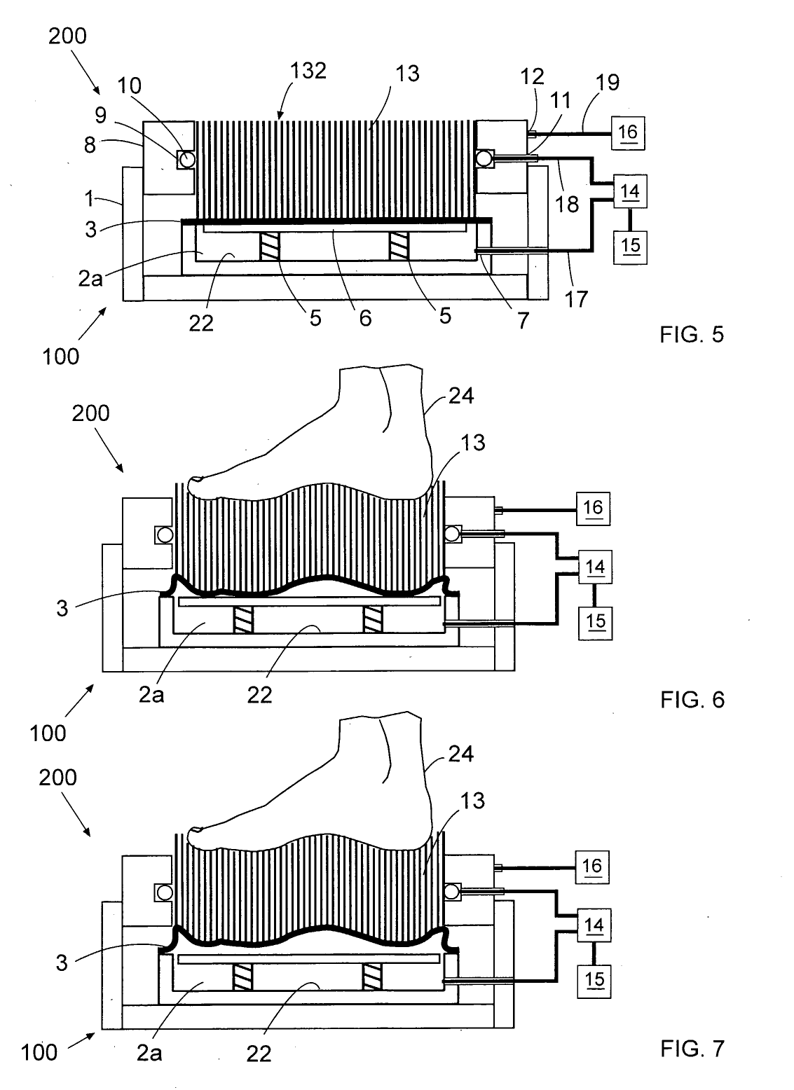

Specifically, according to an embodiment of the invention, the inflation device 100 comprises a base platform 1, a pressure resistant container 2, an elastic membrane 3 (see FIG. 5) sealed to an open face of the container 2, thereby defining a variable volume chamber 2a within the container 2. The inflation device 100 also includes introducing means for reversibly introducing fluid into the chamber 2a. The introducing means include a manifold 17 coupled to the container 2 through an inlet orifice 7 in the base platform 1 and container 2, and a compressor pump 15 arranged with a switch 14 and configured to provide the fluid into the chamber 2a via the manifold 17.

The fluid may be e.g. air, which clearly has no problems of spillage, or of difficulty in filling. Alternatively, a liquid, such as water, could be envisaged. Liquids have the advantage of low compressibility, thus minimizing any adverse effects following failure of any of the components.

Preferably, but not mandatory, the inflation device 100 further comprises a moveably supporting platform 6 mounted within the chamber 2a, substantially parallel to, and biased towards the membrane 3. This platform serves, initially to support the foot, prior to introduction of the fluid. As fluid is introduced into the chamber 2a, the biasing of the platform (e.g. with one or more springs 5) causes the platform 6 to move with the foot, until the foot is completely supported by the fluid-backed membrane 3.

The imprint-forming device 200 includes a bed of elongate rods 13 having substantially equal length, which are mounted vertically in a case 8. In one configuration, the rods can slide freely up, one relative to another, along their length. The rods 13 may be circular in cross-section, or may have cross-section that more closely tessellates, such as square or hexagonal. The use of a tessellating cross-section minimizes the gaps between the rods, with the beneficial effect that any molding compound used to form a replicate is less likely to seep into the bed of rods. When assembled in the apparatus, lower ends 131 of the rods 13 abut the face (upper surface) of the elastic membrane 3. Accordingly, upper ends 132 of the rods 13 form a surface having the same pattern as the upper surface of the elastic membrane 3. In operation, a group of the rods 13 may be depressed by the pressure of the foot sole. Accordingly, when so depressed, the upper ends 132 will take the pattern (i.e., shape) of the plantar surface of the foot representing a negative imprint of the loaded surface of the foot. Accordingly, the lower ends 131 of the rods 13 will form a positive imprint of the loaded surface of the foot.

In order to maintain the created pattern of the plantar surface of the foot the imprint-forming device 200 includes a clamping unit configured for reversibly clamping the bed of rods 13 so that the relative sliding movement of the rods is resisted. According to an embodiment of the imprint-forming device 200, the clamping unit comprises a reversibly deformable tube 10 encircling at least part of the bed of rods 13 that is arranged in a groove 9 formed along the perimeter of the inner wall of the case 8. The tube 10 is coupled to the compressor pump 15 through a manifold 18 passing through an inlet orifice 11 in the case 8.

The reversible clamping can be achieved by manipulation of the pressure of fluid within the tube 10. When the pressure in the tube 10 is increased, the resultant deformation of the tube squeezes the rods together. Use of a device such as this is particularly advantageous, as the same fluid compressor pump 15 used for pumping the chamber 2a can also be used to inflate the tube 10. In this way, production costs can be minimized. In operation, when a foot is placed on the imprint-forming device 200, the compressor pump 15 can first inflate the inflation device 100 for creating an equally distributed load on the plantar surface of the foot uniformly across the whole sole surface. Thereafter, the compressor pump 15 can be switched by the switching device 14 to inflate the tube 10, thereby clamping the bed of rods 13 to resist the relative sliding movement of the rods and maintain the pattern (shape) of the plantar surface of the foot.

Alternatively, the clamping unit can comprise an electromagnet (not shown) acting upon a bed of the rods which should be made of a magnetically susceptible material. In this way, clamping and unclamping can be carried out rapidly, and without the use of fluid systems, which can leak.

Referring to FIG. 14, in a particularly advantageous embodiment, the device further comprises a deformable airtight enclosure 21 surrounding the bed of rods 13, and means configured for reversibly removing air from within the enclosure 21. The means for reversibly removing air from within the enclosure 21 includes a vacuum pump 16 and an enclosure manifold 19 passing through an enclosure orifice 12. The enclosure manifold 19 is configured for coupling the vacuum pump 16 to the enclosure 21. This enclosure, such as a bag made of deformable airtight material, serves inter alia two functions: firstly, when it is “vacuum sealed” around the bed of rods, it serves as an additional or alternative clamping mechanism, enabling the clamped bed of rods to be removed from the imprint-forming device 200 for use as a mould. In this way, a number of rod beds may be used with the same device 200, enabling the device to be used whilst molding is taking place, without needing to replicate the clamping mechanism. Secondly, the enclosure 21 also acts to seal any gaps between the rods down which molding compound might migrate, sticking the rods together and resulting in an uneven surface on the molded item.

Included within the scope of the invention is also a method of replicating the profile of a plantar surface of a foot that employs the apparatus of the present invention. If the apparatus of the invention is not assembled, the method starts from assembling the apparatus. For this purpose, the imprint-forming device 200 is placed on the top of the inflation device 100. If the bed of rods is clamped, the method further includes unclamping the bed of rods, so the rods slide freely.

Referring to FIGS. 6 and 7, in order to fabricate a personally fitted insole, a person (not shown) stands on his foot 24 placed on the bed of the rods 13 of the imprint-forming device 200, thereby the rods 13 are depressed by the pressure of the foot sole. Then, fluid is introduced into the chamber 2a in the amount sufficient to create a predetermined value of pressure within the chamber 2a. As the device 200 is inflated, the pressure on the elastic membrane 3 increases and it is transmitted to the rods and the foot. The vertical position of each rod depends on the elastic characteristics of the soft tissue and the mobility of the foot bones. In this instance, the rods will rise higher in the softer and the more mobile areas of foot and the overall position of the rods will copy the surface shape of the sole with the pressure on its surface evenly balanced.

As specifically shown in FIG. 7, the predetermined value of pressure is established in the chamber 2a is such that the membrane 3 deformed owing to the pressure of the foot is not in contact with an opposite face 22 of the chamber. Accordingly, when the platform (6 in FIG. 5) is present, the predetermined value of pressure is established when the membrane 3 deformed owing to the pressure of the foot is not in contact with the platform 6.

According to an embodiment of the invention, the predetermined value of pressure can be evaluated in advance as the weight of the person divided by the total area of his/her sole surface. For this purpose, the weight of the person and the area of the sole surface can be determined by suitable devices. In order to adjust the pressure in the chamber 2a to the required predetermined value the compressor pump 15 can be equipped with a pressostat (not shown) configured for setting and maintaining the pressure as required.

When the pressure in the chamber 2a is adjusted to the predetermined value, the bed of rods 13 is clamped to resist further sliding. The clamping is achieved by disconnecting the compressor pump 15 from the imprint-forming device 200 by using the switch 14 and connecting the compressor pump 15 to the tube 10 for inflating thereof. As a result of this, the rods 13 will be fixed in a certain position. Thus, the upper surface formed the rod tops represents the negative imprint of the load-bearing surface of the sole, whereas the surface joining the rod bottom ends represents the positive imprint of the foot.

When desired, a flexible liner may conveniently be laid between the clamped bed of rods and molding compound to prevent ingress of the compound to the bed of rods, and to produce a more smooth surface.

When the device also comprises the deformable enclosure 21, the method can further include removing air from the airtight enclosure. The vacuum pump 16 can suck out the air from between the rods 13 and the airtight deformable enclosure 21 will cling to the rods, thus fixing the set in the position obtained as described above. The imprint is now ready for the preparation of the insole. Thereafter, the method includes the step of using either face of the clamped bed of rods as a mould to form a positive or negative replicate of the surface. In this case, the enclosure itself serves the function of the liner, described above.

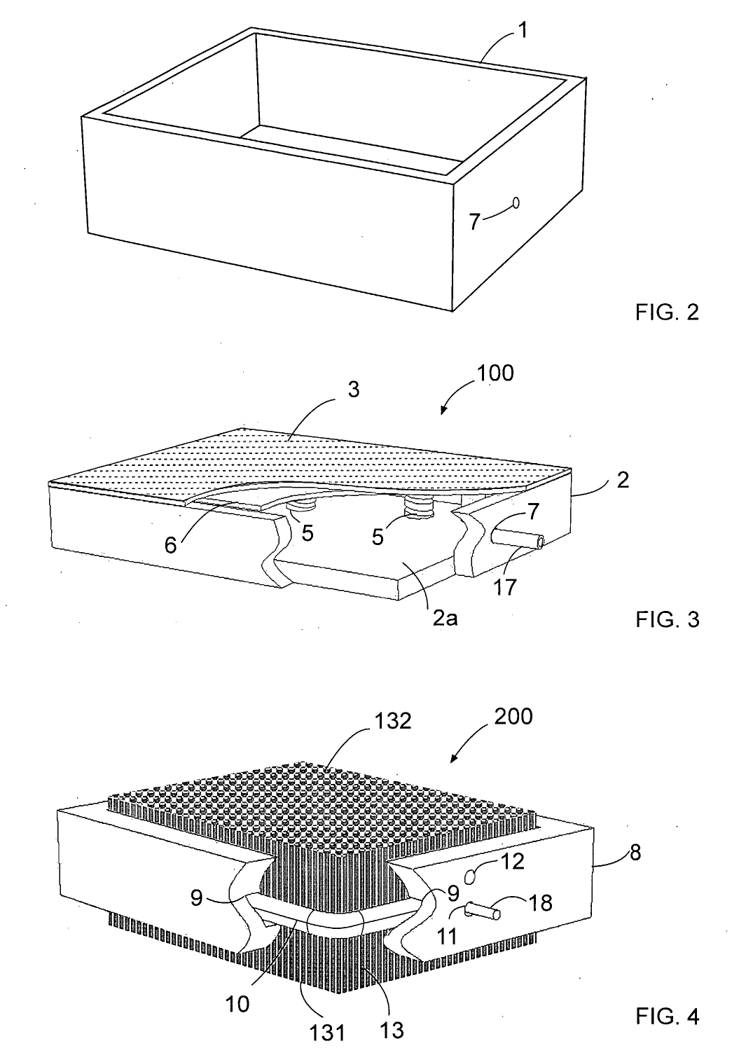

For the preparation of customized insoles, a liquid self-hardening plastic-like material is placed into a vessel similar to the container (1 in FIG. 2). Other molding compounds will be evident to the skilled address for this, and other applications. The positive side of the imprint is lowered down into the solution and left there until solidification of the material. The solidified molding of the insole should be trimmed to the required shape to fit the footwear.

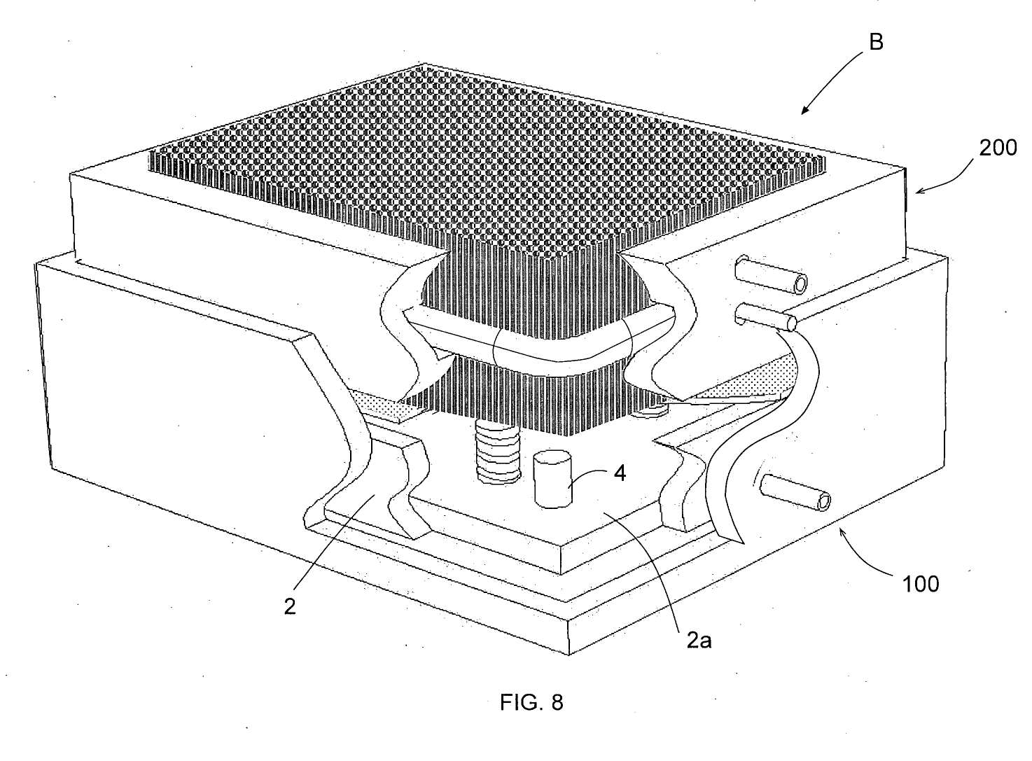

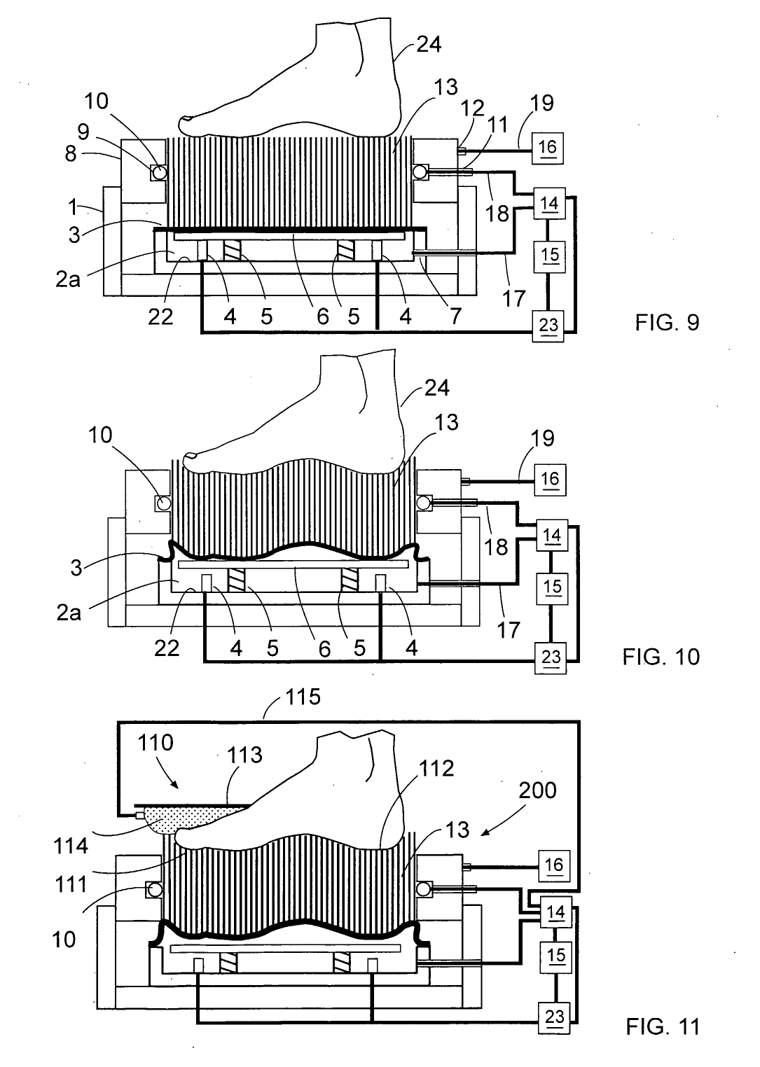

FIG. 8 illustrates a perspective view showing a partial assembly of an apparatus B for replicating the profile of a surface of a sole, according to another embodiment of the invention. In turn, FIGS. 9 and 10 illustrate operation of the apparatus B. According to this embodiment, the inflation device 100 always comprises the moveably mounted platform 6 within the chamber 2a, substantially parallel to, and biased towards the membrane 3. This platform serves initially to support the foot, prior to introduction of the fluid. As fluid is introduced into the chamber, the biasing of the platform 6 (e.g. with one or more springs 5) causes the platform to move with the foot, until the foot 24 is completely supported by the fluid-backed membrane 3. The apparatus B differs from the apparatus (A in FIG. 1) in the fact that the apparatus B further comprises one or more sensing elements 4 arranged in the inflation device 200 and a control unit 23 configured for control of operation of the apparatus B. The sensing elements 4 are configured for sensing contact between the elastic membrane 3 and the opposite face 22 of the container 2 (resulting from deformation of the membrane 3), and forming a contact sensor signal indicating that this contact is broken. The sensing elements 4, in this case, may be conveniently located between the platform 6 and the opposite face of the container 22, and could take the form of a microswitch. Examples of the sensing elements 4 include, but are not limited to, baro-sensors, contact sensors, distance sensors, volume sensors, optical sensors, etc. When required, several such sensors may be employed—for example, on at each corner of the platform—to improve the reliability of the sensing.

The apparatus B operates by introducing fluid into the chamber 2a until the foot 24 is fully supported by the fluid-backed membrane 3, in this way, an even pressure across the face of the object is ensured. The sensing elements 4 act to provide an indication of when this condition is achieved.

The controller unit 23 is electrically coupled to the sensing elements 4, the switching device 14 and the compressor pump 15. The controller unit 23 is responsive to the contact sensor signal and configured to effect the steps of:

(i) unclamping the bed of rods 13 (if clamped);

(ii) receiving the contact sensor signal from the sensing elements 4 indicating contact between the platform 6 and the opposite face 22 of the container 2;

(iii) operating the compressor pump 15 for introducing fluid into the chamber 2a until receiving a signal from the sensing elements 4 indicating loss of contact between the membrane 3 and the platform 6;

(iv) switching the switching device 14 to inflate the tube 10 for clamping the bed of rods 13; and

(v) releasing fluid from the chamber 2a.

In this way, when a user (in the case of making shaped insoles) steps onto the apparatus B, this is sensed by the sensing elements 4. The weight of the user is transferred by the rods onto the supporting platform 6 and sensor elements 4. The control unit 23 (responsive to the sensor signal) then sends a command for introducing fluid (e.g. air, from a compressor pump 15) into the chamber 2a, to inflate the device 100 for providing equally distributed load on the plantar surface of the foot 24.

As the device is inflated, the pressure on the elastic membrane increases and it is transmitted to the rods 13 and the plantar surface of the foot 24. The position of each rod depends on the elastic characteristics of the soft tissue and the mobility of the bones. In this instance, the rods 13 can rise higher in the softer and the more mobile areas of the plantar foot surface and the overall position of the rods will copy the surface of the sole with the pressure on its surface evenly balanced. At this stage, the lowest-positioned rod will cease pressing the platform 6, and the latter will be lifted (see FIG. 10) by the springs 5. The pressure on the sensor elements 4 will be ceased as well. In this case, the control unit 23 will send a command to disconnect the compressor pump 15 from the chamber 2a of inflation device 100, and connect it to the clamping means to inflate the tube 10 and thereby to clamp the bed of rods 13 together in the present position. Thus, the upper surface joining the rod tops represents the negative imprint of the load-bearing surface of the sole and the surface joining the rod bottom ends represents the positive imprint of the foot. Fluid may now be removed from the chamber 2a to reset the apparatus ready for use.

Referring to FIG. 11, a cross-sectional side view of still another embodiment of the apparatus for replicating the profile of a surface of a sole is illustrated. This embodiment differs from that shown in FIGS. 8-10 in the fact that it further includes a forefoot confiner 110 that is designed to prevent a premature raise of the forefoot 111 on the bed of rods 13.

Because the modeling of the bearing area of the sole is carried out in standing position, it is necessary to take into account that the weight is distributed unevenly along the sole area. The main pressure is located at the heel and hindfoot 112 that is part of the foot above the heel. Therefore, the forefoot 111 can rise on the bed of rods 13 before the hindfoot during the inflation of the inflation device 200. This can result in the fact that the insole (not shown), fabricated in accordance with the method of the present invention, can have larger thickness at the front part than at the rear part. To avoid this unfavorable effect, the forefoot confiner 110 includes a holder plate 113 and an inflatable holding cushion 114 arranged between the holder plate 113 and the forefoot 111. The inflatable holding cushion 114 is coupled to the compressor pump 15 through a cushion manifold 115. In operation, when a foot is placed on the imprint-forming device 200, the compressor pump 15 can first inflate the inflation device 100 simultaneously with the holding cushion 114. Thereafter, the compressor pump 15 can be switched by the switching device 14 to inflate the tube 10, thereby clamping the bed of rods 13. When required the forefoot confiner 110 can be controlled by the control unit 23 coupled to the forefoot confiner 110, mutatis mutandis.

The apparatus of the present invention for replicating the profile of a plantar surface of a foot can be adapted for production of personally fitted biomechanical orthopedic insoles for high heel shoes.

While walking on high heels, the body weight is distributed unevenly among the various parts of the feet. The load on the forefoot increases proportionally to the height of the heel, which consequently increases the pressure on the sole of this part of the foot, when compared to the pressure on the sole of the hindfoot.

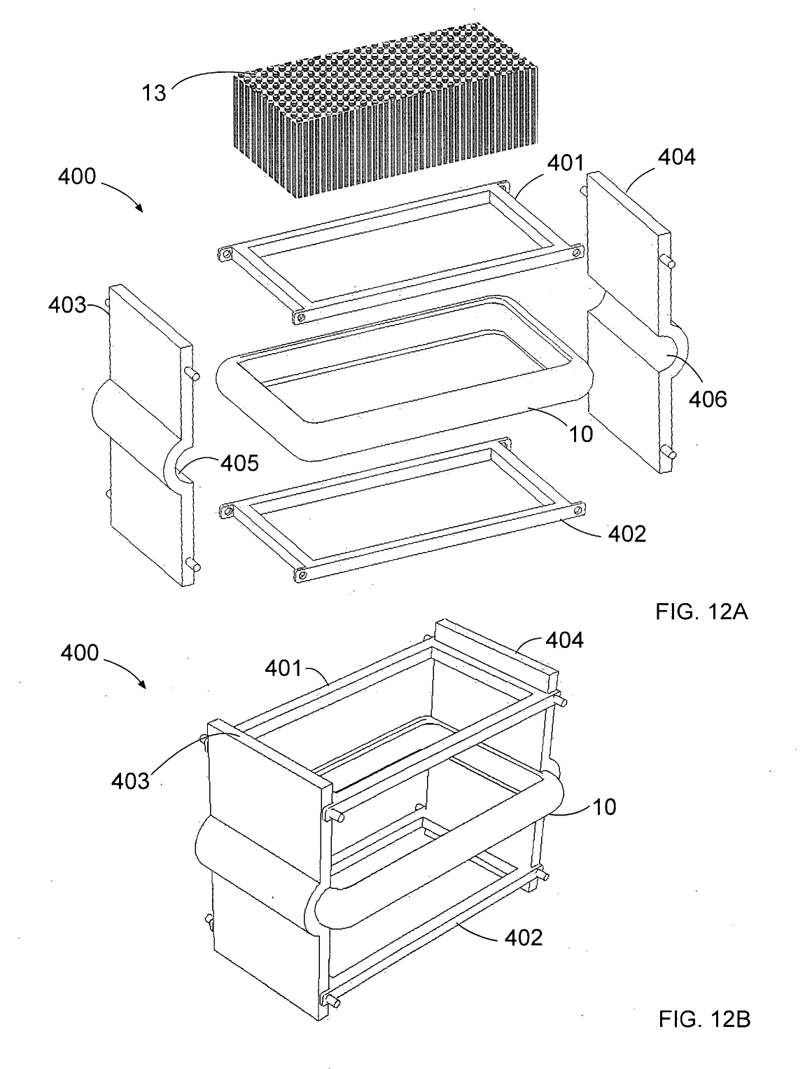

FIGS. 12A and 12B illustrate a perspective exploded and partially assembled views of an embodiment of an imprint-forming device 400 of the apparatus of the present invention designed for replicating the profile of a plantar surface of a foot placed in a condition similar to the condition when the foot is in high heel shoes. The imprint-forming device 400 simulates the conditions occurring when high heel shoes are worn, and can be used for producing personally fitted orthopedic insoles for such shoes. The imprint-forming device 400 includes an upper frame 401 and a lower frame 402 pivotably connected to two lateral walls 403 and 404. This feature allows tilting of the upper frame 401 and the lower frame 402 with respect to the walls 403 and 404 in order to maintain the rods 13 in a vertical position.

The lateral walls 403 and 404 have grooves 405 and 406, correspondingly, formed between the upper and lower frames 401 and 402. The grooves 405 and 406 are arranged for accommodation of the reversibly deformable tube 10 configured for encircling at least part of the bed of rods 13 for clamping and unclamping thereof, when required. Such arrangement of the tube 10 enables free movement of either of the walls 403 or 404 downwards or upwards. It should be understood that alternatively an electromagnet (not shown) can be used for clamping and unclamping the rods 13.

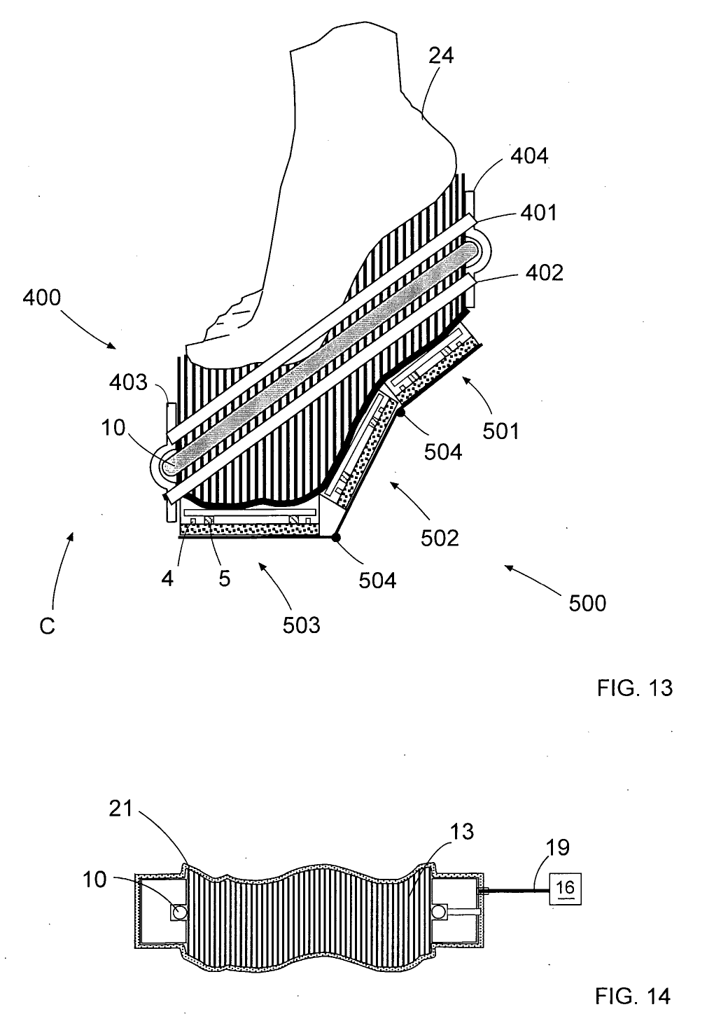

Referring to FIG. 13, the imprint-forming device 400 operates in association with an inflation device 500, thereby forming an apparatus C for replicating the profile of a surface of a sole, according to yet another embodiment of the invention. The inflation device 500 includes three inflation devices 501, 502 and 503 mounted in a common case (not shown) and pivotably connected in series to one another, for example, by means of hinges 504. Each of these inflation devices is similar to the inflation device (100 in FIG. 8). When assembled in the apparatus for replicating the profile of sole surface, the inflation devices 501, 502 and 503 are assigned to correct distribution of the different load created by the various parts of the foot, such as forefoot, midfoot and hindfoot, correspondingly. For the purpose of the present invention, the forefoot refers to the most distal part of the foot, which includes the toes and interphalangeal and metatarsophalangeal joints. The hindfoot refers to the most proximal portion of the foot. This region inter alia includes the following important anatomic structures: the calcaneus, Achilles tendon and the ankle, talocalcaneonavicular, calcaneocuboid and cuneonavicular joints. The midfoot refers to the portion of the foot located between the hindfoot and forefoot. This region includes the subtalar joint, talocalcaneonavicular joint, cuneonavicular, calcaneocuboid, cuboideonavicular, cuneocuboid, intercuneiform and tarsometatarsal joints.

For this purpose, each inflation device is coupled independently to the compressor pump (not shown) and equipped with an independent set of sensing elements 4 determining the optimal specific load for each area of the foot, as described above with reference to FIGS. 8-10.

In operation, when a user stands onto the apparatus C, the imprint-forming device 400 is adjusted to a predetermined foot angle corresponding to the height of the heel of the corresponding high heel shoes (not shown). In turn, the inflation devices 501, 502 and 503 are adjusted is such a manner that the devices 501 is arranged horizontally, whereas the device 503 is tilted at an angle preferably, having the value of the predetermined foot angle. The further operational sequence of the apparatus C is similar to the apparatus B described above with reference to FIGS. 8-10.

As such, those skilled in the art to which the present invention pertains, can appreciate that while the present invention has been described in terms of preferred embodiments, the conception, upon which this disclosure is based, may readily be utilized as a basis for the designing of other structures systems and processes for carrying out the several purposes of the present invention.

Although embodiments of the invention have been described in relation to the production of customized insoles for footwear, it should be understood that the apparatus and method can be used for the profile of any soft uneven surface.

It is to be understood that the phraseology and terminology employed herein are for the purpose of description and should not be regarded as limiting.

It is important, therefore, that the scope of the invention is not construed as being limited by the illustrative embodiments set forth herein. Other variations are possible within the scope of the present invention as defined in the appended claims.

1. An apparatus for replicating the profile of a plantar surface of a foot comprising:
at least one inflation device including:
a pressure resistant container,
an elastic membrane sealed to an open face of said pressure resistant container, thereby defining a variable volume chamber within the container,
a manifold coupled to said pressure resistant container,
a compressor pump configured to provide fluid into the chamber via said manifold, thereby to provide a predetermined pressure within said variable volume chamber, said predetermined pressure required for creating an equally distributed load on said plantar surface of the foot under full load with distribution of the load uniformly across the whole plantar surface;
and
an imprint-forming device mounted on the top of said at least one inflation device and configured for obtaining an imprint of said plantar surface of the foot under the load, said imprint-forming device including:
a bed of elongate rods having substantially equal length,
a clamping unit configured for reversibly clamping the bed of rods so that the relative sliding movement of the rods is resisted, the bed of rods being arranged so that ends of the rods abut the face of said elastic membrane.

2. The apparatus of claim 1 further comprising sensing elements configured for sensing contact between the elastic membrane and an opposite face of the container resulting from deformation of the elastic membrane.

3. The apparatus of claim 1 further comprising a moveably mounted platform within said variable volume chamber, substantially parallel to, and biased towards the elastic membrane.

4. The apparatus of claim 3 further comprising sensing elements configured for sensing contact between the elastic membrane and the platform resulting from deformation of the elastic membrane.

5. The apparatus of claim 1 claims wherein the clamping unit comprises a reversibly deformable tube encircling at least part of the bed of rods, the reversible clamping being achieved by manipulation of the pressure of fluid within the tube, the resultant deformation of the tube squeezes the rods together.

6. The apparatus of claim 1 wherein the clamping unit comprises an electromagnet acting upon a bed of magnetically susceptible rods for reversible clamping thereof.

7. The apparatus of claim 1 further comprising a deformable airtight enclosure surrounding the bed of rods and means to reversibly remove air from within the enclosure.

8. The apparatus of claim 2 further comprising a control unit configured to effect the steps of:
unclamping the bed of rods, if clamped;
receiving a signal from the sensing unit indicating contact between the membrane and the platform;
introducing fluid into the chamber until receiving a signal from the sensing unit indicating loss of contact between the membrane and the platform;
clamping the bed of rods; and
releasing fluid from the chamber.

9. The apparatus of claim 1, comprising three inflation devices pivotably connected in series to one another and assigned to correct distribution of the different load created by forefoot, midfoot and hinfoot, correspondingly.

10. The apparatus of claim 9 wherein said imprint-forming device includes an upper frame and a lower frame pivotably connected to two lateral walls, thereby allowing tilting of the upper frame and the lower frame with respect to the walls in order to maintain the rods in a vertical position.

11. A method for replicating the profile of a plantar surface of a foot using a apparatus according to claim 1, the method comprising:
unclamping the bed of rods, if required, so they slide freely;
pressing the plantar surface against the rods, thus causing deformation of the elastic membrane;
introducing sufficient fluid into the chamber to provide said predetermined pressure within the variable volume chamber;
clamping the bed of rods to resist further sliding;
using either face of the clamped bed of rods as a mould to form a positive or negative replicate of the surface.

12. The method of claim 11 wherein the predetermined value of pressure established in the chamber is such that the membrane deformed owing to the pressure of the foot is not in contact with an opposite face of the chamber.

13. The method of claim 11 wherein the predetermined value of pressure established in the chamber is such that the membrane deformed owing to the pressure of the foot is not in contact with the platform.

14. The method of claim 11 wherein the predetermined value of pressure established in the chamber is evaluated in advance as weight of a user divided by total area of the plantar surface.

15. The method of claim 11 comprising the steps of removing air from a deformable airtight enclosure surrounding the bed of rods.

16. A replicate of the profile of said plantar surface of the foot obtainable by the method of claim 11.

17. A replicate of the profile of said plantar surface of the foot obtainable by the method of claim 12.

18. A replicate of the profile of said plantar surface of the foot obtainable by the method of claim 13.

19. A replicate of the profile of said plantar surface of the foot obtainable by the method of claim 14.

20. A replicate of the profile of said plantar surface of the foot obtainable by the method of claim 15.
